(19) United States
(12) Patent Application Publication
(10) Pub. No.: US 2013/0274282 A1
(43) Pub. Date: Oct. 17, 2013

(54) COMPOSITIONS AND METHODS COMPRISING CELECOXIB OR RELATED COMPOUNDS AND DEXTROMETHORPHAN

(71) Applicant: Herriot Tabuteau, New York, NY (US)

(72) Inventor: Herriot Tabuteau, New York, NY (US)

(21) Appl. No.: 13/857,017

(22) Filed: Apr. 4, 2013

Related U.S. Application Data
(60) Provisional application No. 61/624,694, filed on Apr. 16, 2012.

(51) Int. Cl. A61K 31/415; A61K 31/485

(52) U.S. Cl. CPC A61K 31/415; A61K 31/485
USPC 514/289; 514/406

(57) ABSTRACT

Pain and/or neurological disorders may be treated by administering a therapeutically effective amount of dextromethorphan and a therapeutically effective amount of a compound such as celecoxib that inhibits the metabolism of dextromethorphan, to a person in need thereof. The two compounds may be administered separately, or in a single dosage form or composition as described herein.

### CROSS-REFERENCE TO RELATED APPLICATIONS

This application claims the benefit of U.S. Provisional Application No. 61/624,694, filed on Apr. 16, 2012 under the title “Compositions and Methods Comprising Celecoxib or Related Compounds and Dextromethorphan,” which is expressly incorporated by reference herein in its entirety.

### BACKGROUND

Dextromethorphan is widely used as a cough suppressant and is considered to be safe enough to be sold over the counter. Celecoxib is a nonsteroidal anti-inflammatory agent (NSAID) approved for the treatment of osteoarthritis, rheumatoid arthritis and other conditions.

### SUMMARY

Celecoxib can be used to improve the therapeutic properties, such as pain relieving properties, of dextromethorphan. Celecoxib can be effective in inhibiting or reducing the metabolism of dextromethorphan in some human beings. This may be accomplished by co-administering celecoxib and dextromethorphan.

Some embodiments include a pharmaceutical composition comprising a therapeutically effective amount of dextromethorphan, a therapeutically effective amount of a non-steroidal anti-inflammatory drug (NSAID), such as a cyclooxygenase-2 (COX-2) inhibitor, and a pharmaceutically acceptable excipient.

Some embodiments include a method of treating pain or neurological disorders comprising administering a therapeutically effective amount of dextromethorphan and a therapeutically effective amount of an NSAID, such as a COX-2 inhibitor, to a person in need thereof.

Some embodiments include a method of enhancing the pain relieving properties of dextromethorphan, comprising co-administering dextromethorphan and an NSAID, such as a COX-2 inhibitor.

Some embodiments include a method of increasing dextromethorphan plasma levels in a human being that is an extensive metabolizer of dextromethorphan, comprising co-administering celecoxib and dextromethorphan to the human being.

Some embodiments include a method of inhibiting the metabolism of dextromethorphan, comprising administering celecoxib to a human being, wherein the human being is an extensive metabolizer of dextromethorphan, and wherein dextromethorphan is present in the body of the human being at the same time as celecoxib.

Some embodiments include a method of increasing the metabolic lifetime of dextromethorphan, comprising administering celecoxib to a human being, wherein the human being is an extensive metabolizer of dextromethorphan, and wherein dextromethorphan is present in the body of the human being at the same time as celecoxib.

Some embodiments include a method of correcting extensive metabolism of dextromethorphan, comprising administering celecoxib to a human being in need thereof, such as a human being in need of treatment for pain.

Some embodiments include a method of improving the pain relieving properties of dextromethorphan comprising administering celecoxib in conjunction with administration of dextromethorphan to a human being in need of treatment for pain.

Some embodiments include a method of treating pain comprising administering a combination of celecoxib and dextromethorphan to a human being in need thereof.

### BRIEF DESCRIPTION OF THE DRAWINGS

FIG. 1 depicts the potency (1/IC₅₀) of various NSAIDs for the inhibition of dextromethorphan metabolism.

### DETAILED DESCRIPTION

Dextromethorphan has the structure shown below.

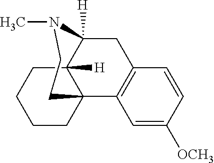 [SMILES: COc1ccc2c(c1)[C@]13CCCC[C@@H]1[C@H](C2)N(C)CC3]

Dextromethorphan is an NMDA receptor antagonist, sigma-1 receptor agonist, and N-type calcium channel blocker having antitussive properties. Because of its mechanism of action, there has been interest from clinicians in using dextromethorphan to treat a variety of neurological conditions including pain. However, results of clinical trials of dextromethorphan as monotherapy for chronic pain have been disappointing. In their review, Weinbroum et al. concluded that the few double-blind human studies of dextromethorphan in chronic and neuropathic pain showed it to be ineffective for the most part (Can J Anesth 2000; 47:585-596.) Gilron et al. reported that dextromethorphan showed little or no analgesic efficacy in their study of patients with facial neuralgias (Neurology 2000; 55:964-971). Similarly, dextromethorphan did not reduce pain significantly more than placebo in Sang et al.'s study of patients with diabetic neuropathy and postherpetic neuralgia (Anesthesiology 2002; 96:1053-1061).

The disappointing clinical effects observed with dextromethorphan monotherapy in clinical trials may be associated with its rapid metabolism in the human liver. This rapid hepatic metabolism limits systemic drug exposure in individuals who are extensive metabolizers. Human beings can be: 1) extensive metabolizers of dextromethorphan—those who rapidly metabolize dextromethorphan; 2) poor metabolizers of dextromethorphan—those who only poorly metabolize dextromethorphan; or 3) intermediate metabolizers of dextromethorphan—those whose metabolism of dextromethorphan is somewhere between that of an extensive metabolizer and a poor metabolizer. Extensive metabolizers of dextromethorphan are a significant portion of the human population.

When given the same oral dose of dextromethorphan, plasma levels of dextromethorphan are significantly higher in poor metabolizers or intermediate metabolizers as compared to extensive metabolizers of dextromethorphan. The clearance of dextromethorphan for extensive metabolizers is believed to be about 110 L/min. This high rate of clearance can significantly reduce plasma concentrations of dextromethorphan even at high doses. The low plasma concentrations of dextromethorphan can limit its clinical utility as a single agent for extensive metabolizers, and possibly intermediate metabolizers, of dextromethorphan. Some NSAIDs and COX-2 inhibitors, such as celecoxib, inhibit the metabolism of dextromethorphan, and can thus improve its therapeutic efficacy.

Pain or neurological disorders may be treated by a method comprising administering a therapeutically effective amount of dextromethorphan and a therapeutically effective amount of an NSAID, such as a COX-2 inhibitor, to a person in need thereof.

Pain relieving properties of dextromethorphan may be enhanced by a method comprising co-administering dextromethorphan and an NSAID, including a COX-2 inhibitor such as celecoxib, with dextromethorphan.

These methods may be used to treat, or provide relief to, any type of pain including, but not limited to, musculoskeletal pain, neuropathic pain, cancer-related pain, acute pain, nociceptive pain, etc.

Examples of musculoskeletal pain include low back pain (i.e. lumbosacral pain), primary dysmenorrhea, and arthritic pain, such as pain associated with rheumatoid arthritis, juvenile rheumatoid arthritis, osteoarthritis, axial spondyloarthritis including ankylosing spondylitis, etc.

In some embodiments, a combination of dextromethorphan and an NSAID such as celecoxib is used to treat chronic musculoskeletal pain.

Examples of neuropathic pain include diabetic peripheral neuropathy, post-herpetic neuralgia, trigeminal neuralgia, monoradiculopathies, phantom limb pain, central pain, etc. Other causes of neuropathic pain include cancer-related pain, lumbar nerve root compression, spinal cord injury, post-stroke pain, central multiple sclerosis pain, HIV-associated neuropathy, and radio- or chemo-therapy associated neuropathy, etc.

The term “treating” or “treatment” includes the diagnosis, cure, mitigation, treatment, or prevention of disease in man or other animals, or any activity that otherwise affects the structure or any function of the body of man or other animals.

Any compound that inhibits the metabolism of dextromethorphan may be used in combination with dextromethorphan to improve the therapeutic properties of dextromethorphan. Some compounds that inhibit the metabolism of dextromethorphan may include, but are not limited to, NSAIDs such as celecoxib, non-celecoxib NSAIDs, or metabolites thereof. Dextromethorphan and the compound that inhibits dextromethorphan metabolism may be administered in separate compositions or dosage forms, or may be administered in a single composition or dosage form comprising both.

Celecoxib is a COX-2 inhibitor, a type of NSAID, which possesses anti-inflammatory and anti-nociceptive properties. Celecoxib has the structure shown below.

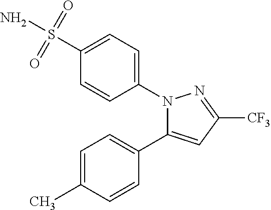 [SMILES: Cc1ccc(-c2cc(C(F)(F)F)nn2-c2ccc(S(N)(=O)=O)cc2)cc1]

Combining celecoxib with dextromethorphan may provide greater efficacy, such as greater pain relief, than would otherwise be achieved by administering either component alone. In extensive metabolizers, dextromethorphan can be rapidly and extensively metabolized, yielding low systemic exposure even at high doses. Celecoxib, besides being an anti-inflammatory and analgesic agent, is an inhibitor of dextromethorphan metabolism. Celecoxib has an IC50 of 2.6-2.9 μM for the demethylation of dextromethorphan, as demonstrated herein. The IC50 is also referred to as the half maximal inhibitory concentration, and represents the concentration of drug, such as celecoxib, needed to inhibit the metabolism of dextromethorphan by half. As explained above, this inhibition may augment dextromethorphan plasma levels, resulting in additive or synergistic pain relief. Thus, while inhibition of dextromethorphan metabolism is only one of many potential benefits of the combination, co-administration of dextromethorphan with celecoxib may thereby enhance the analgesic properties of celecoxib for many individuals.

Many types of pain arise from both nociceptive and neuropathic pathophysiologic mechanisms. A combination of celecoxib and dextromethorphan may therefore be advantageous for treating many pain conditions since celecoxib may preferentially target the nociceptive and inflammatory components while dextromethorphan may preferentially address the neuropathic component of the pain condition.

Co-administering dextromethorphan and an NSAID, such as celecoxib, does not necessarily require that the two compounds be administered in the same dosage form. For example, the two compounds may be administered in a single dosage form, or they may be administered in two separate dosage forms. Additionally, the two compounds may be administered at the same time, but this is not required. The compounds can be given at different times as long as both are in a human body at the same time for at least a portion of the time that treatment by co-administration is being carried out.

In some embodiments, co-administration of a combination of celecoxib and dextromethorphan results in both celecoxib and dextromethorphan contributing to the pain relieving properties of the combination. For example, the combination may have improved pain relieving properties as compared to celecoxib alone or compared to dextromethorphan alone.

In some embodiments, the combination may have improved pain relieving properties of at least about 0.5%, at least about 1%, at least about 10%, at least about 20%, at least about 30%, at least about 50%, at least 100%; up to about 500% or up to 1000%; and/or about 0.5% to about 1000% as compared to celecoxib alone.

In some embodiments, the combination may have improved pain relieving properties of at least about 0.5%, at least about 1%, at least about 10%, at least about 20%, at least about 30%, at least about 50%, at least 100%; up to about 500% or up to 1000%; and/or about 0.5% to about 1000% as compared to dextromethorphan alone.

Unless otherwise indicated, any reference to a compound herein such as dextromethorphan or celecoxib by structure, name, or any other means, includes pharmaceutically acceptable salts; alternate solid forms, such as polymorphs, solvates, hydrates, etc.; tautomers; or any other chemical species that may rapidly convert to a compound described herein under conditions in which the compounds are used as described herein.

A dosage form or a composition may be a blend or mixture of dextromethorphan and a compound that inhibits metabolism of dextromethorphan, such as celecoxib, either alone or within a vehicle. For example, dextromethorphan and celecoxib may be dispersed within each other or dispersed together within a vehicle. A dispersion may include a mixture of solid materials wherein small individual particles are substantially one compound, but the small particles are dispersed within one another, such as might occur if two powders of two different drugs are blended with a solid vehicle material, and the blending is done in the solid form. In some embodiments, dextromethorphan and celecoxib may be substantially uniformly dispersed within a composition or dosage form. Alternatively, dextromethorphan and celecoxib may be in separate domains or phases within a composition or dosage form. For example, one drug may be in a coating and another drug may be in a core within the coating.

Dextromethorphan and/or a compound that inhibits the metabolism of dextromethorphan, such as an NSAID, including a COX-2 inhibitor such as celecoxib (all of which are referred to collectively herein as “therapeutic compounds” for convenience) may be combined with a pharmaceutical carrier selected on the basis of the chosen route of administration and standard pharmaceutical practice as described, for example, in Remington's Pharmaceutical Sciences, 2005, the disclosure of which is hereby incorporated herein by reference, in its entirety. The relative proportions of active ingredient and carrier may be determined, for example, by the solubility and chemical nature of the compounds, chosen route of administration and standard pharmaceutical practice.

Therapeutic compounds may be administered by any means that may result in the contact of the active agent(s) with the desired site or site(s) of action in the body of a patient. The compounds may be administered by any conventional means available for use in conjunction with pharmaceuticals, either as individual therapeutic agents or in a combination of therapeutic agents. For example, they may be administered as the sole active agents in a pharmaceutical composition, or they can be used in combination with other therapeutically active ingredients.

Therapeutic compounds may be administered to a human patient in a variety of forms adapted to the chosen route of administration, e.g., orally or parenterally. Parenteral administration in this respect includes administration by the following routes: intravenous, intramuscular, subcutaneous, intraocular, intrasynovial, transepithelial including transdermal, ophthalmic, sublingual and buccal; topically including ophthalmic, dermal, ocular, rectal and nasal inhalation via insufflation, aerosol and rectal systemic.

The ratio of dextromethorphan to celecoxib may vary. In some embodiments, the weight ratio of dextromethorphan to celecoxib may be about 0.1 to about 2, about 0.2 to about 1, about 0.1 to about 0.3, about 0.2 to about 0.4, about 0.3 to about 0.5, about 0.5 to about 0.7, about 0.8 to about 1, about 0.2, about 0.3, about 0.4, about 0.45, about 0.6, about 0.9, or any ratio in a range bounded by, or between, any of these values. A ratio of 0.1 indicates that the weight of dextromethorphan is 1/10 that of celecoxib. A ratio of 2 indicates that the weight of dextromethorphan is 2 times that of celecoxib.

The amount of dextromethorphan in a therapeutic composition may vary. For example, some liquid compositions may comprise about 0.0001% (w/v) to about 50% (w/v), about 0.01% (w/v) to about 20% (w/v), about 0.01% to about 10% (w/v), about 0.001% (w/v) to about 1% (w/v), about 0.1% (w/v) to about 0.5% (w/v), about 1% (w/v) to about 3% (w/v), about 3% (w/v) to about 5% (w/v), about 5% (w/v) to about 7% (w/v), about 7% (w/v) to about 10% (w/v), about 10% (w/v) to about 15% (w/v), about 15% (w/v) to about 20% (w/v), about 20% (w/v) to about 30% (w/v), about 30% (w/v) to about 40% (w/v), or about 40% (w/v) to about 50% (w/v) of dextromethorphan.

Some liquid dosage forms may contain about 20 mg to about 500 mg, about 30 mg to about 350 mg, about 50 mg to about 200 mg, about 50 mg to about 70 mg, about 80 mg to about 100 mg, about 110 mg to about 130 mg, about 170 mg to about 190 mg, about 60 mg, about 90 mg, about 120 mg, or about 180 mg of dextromethorphan, or any amount of dextromethorphan in a range bounded by, or between, any of these values.

Some solid compositions may comprise at least about 5% (w/w), at least about 10% (w/w), at least about 20% (w/w), at least about 50% (w/w), at least about 70% (w/w), at least about 80%, about 10% (w/w) to about 30% (w/w), about 10% (w/w) to about 20% (w/w), about 20% (w/w) to about 30% (w/w), about 30% (w/w) to about 50% (w/w), about 30% (w/w) to about 40% (w/w), about 40% (w/w) to about 50% (w/w), about 50% (w/w) to about 80% (w/w), about 50% (w/w) to about 60% (w/w), about 70% (w/w) to about 80% (w/w), or about 80% (w/w) to about 90% (w/w) of dextromethorphan.

Some solid dosage forms may contain about 20 mg to about 500 mg, about 30 mg to about 350 mg, about 50 mg to about 200 mg, about 50 mg to about 70 mg, about 80 mg to about 100 mg, about 110 mg to about 130 mg, about 170 mg to about 190 mg, about 60 mg, about 90 mg, about 120 mg, or about 180 mg of dextromethorphan, or any amount of dextromethorphan in a range bounded by, or between, any of these values.

The amount of celecoxib in a therapeutic composition may vary. For example, some liquid compositions may comprise about 0.0001% (w/v) to about 50% (w/v), about 0.01% (w/v) to about 20% (w/v), about 0.01% to about 10% (w/v), about 1% (w/v) to about 3% (w/v), about 3% (w/v) to about 5% (w/v), about 5% (w/v) to about 7% (w/v), about 5% (w/v) to about 15% (w/v), about 7% (w/v) to about 10% (w/v), about 10% (w/v) to about 15% (w/v), about 15% (w/v) to about 20% (w/v), about 20% (w/v) to about 30% (w/v), about 30% (w/v) to about 40% (w/v), or about 40% (w/v) to about 50% (w/v) of celecoxib.

Some liquid dosage forms may contain about 50 mg to about 1000 mg, about 200 mg to about 300 mg, about 180 mg to about 220 mg, about 280 mg to about 320 mg, about 200 mg, or about 300 mg of celecoxib, or any amount of celecoxib in a range bounded by, or between, any of these values.

Some solid compositions may comprise at least about 5% (w/w), at least about 10% (w/w), at least about 20% (w/w), at least about 50% (w/w), at least about 70% (w/w), at least about 80%, about 10% (w/w) to about 30% (w/w), about 10% (w/w) to about 20% (w/w), about 20% (w/w) to about 30% (w/w), about 30% (w/w) to about 50% (w/w), about 30% (w/w) to about 40% (w/w), about 40% (w/w) to about 50% (w/w), about 50% (w/w) to about 80% (w/w), about 50% (w/w) to about 60% (w/w), about 70% (w/w) to about 80% (w/w), or about 80% (w/w) to about 90% (w/w) of celecoxib.

Some solid dosage forms may contain about 50 mg to about 1000 mg, about 200 mg to about 300 mg, about 180 mg to about 220 mg, about 280 mg to about 320 mg, about 200 mg, or about 300 mg of celecoxib, or any amount of celecoxib in a range bounded by, or between, any of these values.

In some embodiments, celecoxib is administered at a dose that results in a celecoxib plasma level of about 1 μM to about 10 μM, about 1 μM to about 5 μM, about 2 μM to about 3 μM, or about 2.8 μM to about 3 μM, about 1.5 μM to about 2 μm, about 4.5 μM to about 5 μM, about 2.5 μM to about 3 μM, about 1.8 μM, about 4.8 μM, about 2.9 μM, or about 2.8 μM.

For compositions comprising both dextromethorphan and celecoxib, some liquids may comprise about 0.0001% (w/v) to about 50% (w/v), about 0.01% (w/v) to about 20% (w/v), about 0.01% to about 10% (w/v), about 1% (w/v) to about 3% (w/v), about 3% (w/v) to about 5% (w/v), about 5% (w/v) to about 7% (w/v), about 5% (w/v) to about 15% (w/v), about 7% (w/v) to about 10% (w/v), about 10% (w/v) to about 15% (w/v), about 15% (w/v) to about 20% (w/v), about 20% (w/v) to about 30% (w/v), about 30% (w/v) to about 40% (w/v), or about 40% (w/v) to about 50% (w/v) of dextromethorphan and celecoxib combined. Some solid compositions may comprise at least about 5% (w/w), at least about 10% (w/w), at least about 20% (w/w), at least about 50% (w/w), at least about 70% (w/w), at least about 80%, about 10% (w/w) to about 30% (w/w), about 10% (w/w) to about 20% (w/w), about 20% (w/w) to about 30% (w/w), about 30% (w/w) to about 50% (w/w), about 30% (w/w) to about 40% (w/w), about 40% (w/w) to about 50% (w/w), about 50% (w/w) to about 80% (w/w), about 50% (w/w) to about 60% (w/w), about 70% (w/w) to about 80% (w/w), or about 80% (w/w) to about 90% (w/w) of dextromethorphan and celecoxib combined. In some embodiments, the weight ratio of dextromethorphan to celecoxib in a single composition or dosage form may be about 0.1 to about 2, about 0.2 to about 1, about 0.1 to about 0.3, about 0.2 to about 0.4, about 0.3 to about 0.5, about 0.5 to about 0.7, about 0.8 to about 1, about 0.2, about 0.3, about 0.4, about 0.45, about 0.6, about 0.9, or any ratio in a range bounded by, or between, any of these values.

A therapeutically effective amount of a therapeutic compound may vary depending upon the circumstances. For example, a daily dose of dextromethorphan may in some instances range from about 0.1 mg to about 1000 mg, about 40 mg to about 1000 mg, about 20 mg to about 600 mg, about 60 mg to about 700 mg, about 100 mg to about 400 mg, about 20 mg to about 60 mg, about 60 mg to about 100 mg, about 100 mg to about 200 mg, about 100 mg to about 140 mg, about 160 mg to about 200 mg, about 200 mg to about 300 mg, about 220 mg to about 260 mg, about 300 mg to about 400 mg, about 340 mg to about 380 mg, about 400 mg to about 500 mg, or about 500 mg to about 600 mg, about 120 mg, about 180 mg, about 240 mg, about 360 mg, or any daily dose in a range bounded by, or between, any of these values. Dextromethorphan may be administered once daily, or twice daily or every 12 hours in amount that is about half of the daily dose.

A daily dose of celecoxib, may in some instances range from about 10 mg to about 1000 mg, about 50 mg to about 600 mg, about 100 mg to about 2000 mg, about 50 mg to about 100 mg, about 100 mg to about 200 mg, about 200 mg about 300 mg, about 300 mg to about 400 mg, about 400 mg to about 500 mg, about 400 mg to about 600 mg, about 360 mg to about 440 mg, about 560 mg to about 640 mg, or about 500 mg to about 600 mg, about 400 mg, about 600 mg, or any daily dose in a range bounded by, or between, any of these values. Celecoxib may be administered once daily, or twice daily or every 12 hours in amount that is about half of the daily dose.

Therapeutic compounds may be formulated for oral administration, for example, with an inert diluent or with an edible carrier, or it may be enclosed in hard or soft shell gelatin capsules, compressed into tablets, or incorporated directly with the food of the diet. For oral therapeutic administration, the active compound may be incorporated with an excipient and used in the form of ingestible tablets, buccal tablets, troches, capsules, elixirs, suspensions, syrups, wafers, and the like.

Tablets, troches, pills, capsules and the like may also contain one or more of the following: a binder such as gum tragacanth, acacia, corn starch or gelatin; an excipient, such as dicalcium phosphate; a disintegrating agent such as corn starch, potato starch, alginic acid and the like; a lubricant such as magnesium stearate; a sweetening agent such as sucrose, lactose or saccharin; or a flavoring agent such as peppermint, oil of wintergreen or cherry flavoring. When the dosage unit form is a capsule, it may contain, in addition to materials of the above type, a liquid carrier. Various other materials may be present as coating, for instance, tablets, pills, or capsules may be coated with shellac, sugar or both. A syrup or elixir may contain the active compound, sucrose as a sweetening agent, methyl and propylparabens as preservatives, a dye and flavoring, such as cherry or orange flavor. It may be desirable for material in a dosage form or pharmaceutical composition to be pharmaceutically pure and substantially non toxic in the amounts employed.

Some compositions or dosage forms may be a liquid, or may comprise a solid phase dispersed in a liquid.

Therapeutic compounds may be formulated for parental or intraperitoneal administration. Solutions of the active compounds as free bases or pharmacologically acceptable salts can be prepared in water suitably mixed with a surfactant, such as hydroxypropylcellulose. A dispersion can also have an oil dispersed within, or dispersed in, glycerol, liquid polyethylene glycols, and mixtures thereof. Under ordinary conditions of storage and use, these preparations may contain a preservative to prevent the growth of microorganisms.

Compositions or dosage forms may be intended for sustained or immediate release, depending upon the particular need. In some embodiments, a dosage form or composition may release dextromethorphan within about 0.5 hours, about 1 hour, about 2 hours, about 3 hours, about 4 hours, or about 6 hours of administration. Some dosage forms or compositions may release celecoxib within about 0.5 hours, about 1 hour, about 2 hours, about 3 hours, about 4 hours, or about 6 hours of administration. Some dosage forms or compositions may release both dextromethorphan and celecoxib within about 0.5 hours, about 1 hour, about 2 hours, about 3 hours, about 4 hours, or about 6 hours of administration.

It may be helpful for dextromethorphan and celecoxib to be released in such a manner that that the relative amounts of the two compounds in a person's system are at least somewhat constant. Thus, it may be desirable for a dosage form or a composition to release dextromethorphan and celecoxib at a substantially constant ratio from about the time of administration to a person until at least about 10%, about 25%, about 50%, about 75%, or about 90% of both drugs have been released from the dosage form or composition.

The combination of celecoxib and dextromethorphan may be sufficiently effective that additional pain relieving medications, such as paracetamol or acetaminophen; steroids such as dexamethasone; γ-aminobutyric acid (GABA) analogs such as gabapentin; benzodiazepines such as triazolam; or opiates are not required to treat pain. As a result, nausea and vomiting may not be a problem, and drugs such as serotonin 5-HT3 receptor antagonists, for example ondansetron, may not be required.

In some embodiments related to the treatment of pain, the person receiving treatment receives substantially no paracetamol. In some embodiments, a dosage form is substantially free of paracetamol.

In some embodiments related to the treatment of pain, the person receiving treatment receives substantially no dexamethasone. In some embodiments, a dosage form is substantially free of dexamethasone.

In some embodiments related to the treatment of pain, the person receiving treatment receives substantially no gabapentin. In some embodiments, a dosage form is substantially free of gabapentin.

In some embodiments related to the treatment of pain, the person receiving treatment receives substantially no triazolam. In some embodiments, a dosage form is substantially free of triazolam.

In some embodiments related to the treatment of pain, the person receiving treatment receives substantially no ondansetron. In some embodiments, a dosage form is substantially free of ondansetron.

For methods related to the treatment of pain, compounds typically used for treating cold and flu-like symptoms may not be required, thus antihistamines such as chlorpheniramine maleate, and decongestants such as pseudoephedrine hydrochloride, may not be required.

In some embodiments related to the treatment of pain, the person receiving treatment receives substantially no chlorpheniramine maleate. In some embodiments, a dosage form is substantially free of chlorpheniramine maleate.

In some embodiments related to the treatment of pain, the person receiving treatment receives substantially no pseudoephedrine hydrochloride. In some embodiments, a dosage form is substantially free of pseudoephedrine hydrochloride.

### SPECIFICALLY CONTEMPLATED EMBODIMENTS

The following are examples of embodiments that are specifically contemplated by the inventor:

### Embodiment A1

A pharmaceutical composition comprising a therapeutically effective amount of dextromethorphan and a therapeutically effective amount of a compound that inhibits the metabolism of dextromethorphan and a pharmaceutically acceptable excipient.

### Embodiment A2

A method of treating pain or neurological disorders comprising administering a therapeutically effective amount of dextromethorphan and a therapeutically effective amount of a compound that inhibits the metabolism of dextromethorphan, to a person in need thereof.

### Embodiment A3

A method of enhancing the pain relieving properties of dextromethorphan, comprising co-administering dextromethorphan and a compound that inhibits the metabolism of dextromethorphan.

### Embodiment A4

The method of embodiment A2 or A3, wherein the dextromethorphan and the compound that inhibits the metabolism of dextromethorphan are administered in separate dosage forms.

### Embodiment A5

The method of embodiment A4, wherein the pain comprises postoperative pain, cancer pain, arthritic pain, lumbosacral pain, musculoskeletal pain, nociceptive pain, or neuropathic pain.

### Embodiment A6

The method of embodiment A5, wherein the pain comprises postoperative pain.

### Embodiment A7

The method of embodiment A5, wherein the pain comprises cancer pain.

### Embodiment A8

The method of embodiment A5, wherein the pain comprises arthritic pain.

### Embodiment A9

The method of embodiment A5, wherein the pain comprises lumbosacral pain.

### Embodiment A10

The method of embodiment A5, wherein the pain comprises musculoskeletal pain.

### Embodiment A11

The method of embodiment A5, wherein the pain comprises neuropathic pain.

### Embodiment A12

The method of embodiment A5, wherein the pain comprises nociceptive pain.

### Embodiment A13

The composition or method of any one of embodiments A1-12, wherein the compound that inhibits the metabolism of dextromethorphan is celecoxib, or a metabolite thereof.

### Embodiment A14

The composition or method of embodiment A13, wherein the compound that inhibits the metabolism of dextromethorphan is celecoxib.

### Embodiment A15

The composition or method of any one of embodiments A1-12, wherein the compound that inhibits the metabolism of dextromethorphan is a non-celecoxib NSAID.

### Embodiment A16

The composition of any one of embodiments A1 and A13-15, wherein the composition is a liquid or comprises a solid phase dispersed in a liquid.

### Embodiment A17

The composition of embodiment A16, wherein the concentration of dextromethorphan is about 0.01% (w/v) to about 10% (w/v).

### Embodiment A18

The composition of any one of embodiments A1 and A13-15, wherein the composition is a solid and the amount of dextromethorphan is at least about 10% (w/w).

### Embodiment A19

The composition of any one of embodiments A1 and A13-18, wherein the dextromethorphan and the compound that inhibits the metabolism of dextromethorphan are dispersed within each other or dispersed together within a vehicle.

### Embodiment A20

The composition of any one of embodiments A1 and A13-19, wherein the dextromethorphan and the compound that inhibits the metabolism of dextromethorphan are substantially uniformly dispersed within the composition.

### Embodiment A21

A dosage form comprising a composition according to any one of embodiments A1 and A13-20, wherein the dosage form releases the dextromethorphan and the compound that inhibits the metabolism of dextromethorphan at a substantially constant ratio after administration to a person until at least about 50% of both drugs have been released from the dosage form.

### Embodiment A22

A dosage form comprising a composition according to any one of embodiments A1 and A13-20, wherein the dosage form releases the dextromethorphan and the compound that inhibits the metabolism of dextromethorphan within about 4 hours of administration.

### Embodiment B1

A method of increasing dextromethorphan plasma levels in a human being that is an extensive metabolizer of dextromethorphan, comprising co-administering celecoxib to the human being receiving a treatment that includes administration of dextromethorphan.

### Embodiment B2

A method of inhibiting metabolism of dextromethorphan, comprising administering celecoxib to a human being, wherein the human being is an extensive metabolizer of dextromethorphan, and wherein dextromethorphan is present in the body of the human being at the same time as celecoxib.

### Embodiment B3

A method of increasing the metabolic lifetime of dextromethorphan, comprising administering celecoxib to a human being, wherein the human being is an extensive metabolizer of dextromethorphan, and wherein dextromethorphan is present in the body of the human being at the same time as celecoxib.

### Embodiment B4

A method of correcting extensive metabolism of dextromethorphan, comprising administering celecoxib to a human being in need thereof.

### Embodiment B5

The method of any of embodiments B1-4, wherein dextromethorphan is administered to the human being for the treatment of pain.

### Embodiment B6

A method of improving pain relieving properties of dextromethorphan comprising administering celecoxib in conjunction with administration of dextromethorphan to a human being in need of treatment for pain.

### Embodiment B7

A method of treating pain comprising administering a combination of celecoxib and dextromethorphan to a human being in need thereof.

### Embodiment B8

The method of any of embodiments B5-7, wherein the pain comprises postoperative pain, cancer pain, arthritic pain, lumbosacral pain, musculoskeletal pain, central multiple sclerosis pain, nociceptive pain, or neuropathic pain.

### Embodiment B9

The method of embodiment B8, wherein the pain comprises postoperative pain.

### Embodiment B10

The method of embodiment B8, wherein the pain comprises cancer pain.

### Embodiment B11

The method of embodiment B8, wherein the pain comprises arthritic pain.

### Embodiment B12

The method of embodiment B8, wherein the pain comprises lumbosacral pain.

### Embodiment B13

The method of embodiment B8, wherein the pain comprises musculoskeletal pain.

### Embodiment B14

The method of embodiment B8, wherein the pain comprises chronic musculoskeletal pain.

### Embodiment B15

The method of embodiment B8, wherein the pain comprises neuropathic pain.

### Embodiment B16

The method of embodiment B8, wherein the pain comprises nociceptive pain.

### Embodiment B17

The method of any of embodiments B4-16, wherein substantially no paracetamol is administered to the human being.

### Embodiment B18

The method of any of embodiments B4-16, wherein substantially no dexamethasone is administered to the human being.

### Embodiment B19

The method of any of embodiments B4-16, wherein substantially no gabapentin is administered to the human being.

### Embodiment B20

The method of any of embodiments B4-16, wherein substantially no triazolam is administered to the human being.

### Embodiment B21

The method of any of embodiments B4-16, wherein substantially no ondansetron is administered to the human being.

### Embodiment B22

The method of any of embodiments B4-16, wherein substantially no chlorpheniramine maleate is administered to the human being.

### Embodiment B23

The method of any of embodiments B4-16, wherein substantially no pseudoephedrine hydrochloride is administered to the human being.

### Embodiment B24

An oral dosage form comprising at least 20 mg of dextromethorphan and an effective amount of celecoxib to inhibit metabolism in a human being that is an extensive metabolizer of dextromethorphan.

### Embodiment B25

The oral dosage form of embodiment B24, wherein about 30 mg to about 350 mg of dextromethorphan is present in the dosage form.

### Embodiment B26

The oral dosage form of embodiment B24 or B25, wherein about 100 mg to about 400 mg of celecoxib is present in the dosage form.

### Embodiment B27

The oral dosage form of any of embodiments B24-26, which is substantially free of paracetamol.

### Embodiment B28

The oral dosage form of any of embodiments B24-27, which is substantially free of dexamethasone.

### Embodiment B29

The oral dosage form of any of embodiments B24-28, which is substantially free of gabapentin.

### Embodiment B30

The oral dosage form of any of embodiments B24-29, which is substantially free of triazolam.

### Embodiment B31

The oral dosage form of any of embodiments B24-30, which is substantially free of ondansetron.

### Embodiment B32

The oral dosage form of any of embodiments B24-31, which is substantially free of pseudoephedrine hydrochloride.

### Embodiment B33

The method of any of embodiments B5-32, wherein the pain comprises musculoskeletal pain, neuropathic pain, cancer-related pain, acute pain, or nociceptive pain.

### Embodiment B34

The method of Embodiment B33, wherein the pain is associated with rheumatoid arthritis.

### Embodiment B35

The method of Embodiment B33, wherein the pain is associated with juvenile rheumatoid arthritis.

### Embodiment B36

The method of Embodiment B33, wherein the pain is associated with osteoarthritis.

### Embodiment B37

The method of Embodiment B33, wherein the pain is associated with an axial spondyloarthritis.

### Embodiment B38

The method of Embodiment B33, wherein the pain is associated with ankylosing spondylitis.

### Embodiment B39

The method of Embodiment B33, wherein the pain is associated with diabetic peripheral neuropathy.

### Embodiment B40

The method of Embodiment B33, wherein the pain is associated with post-herpetic neuralgia.

### Embodiment B41

The method of Embodiment B33, wherein the pain is associated with trigeminal neuralgia.

### Embodiment B42

The method of Embodiment B33, wherein the pain is associated with monoradiculopathies.

### Embodiment B43

The method of Embodiment B33, wherein the pain is associated with phantom limb pain.

### Embodiment B44

The method of Embodiment B33, wherein the pain is associated with central pain.

### Embodiment B45

The method of Embodiment B33, wherein the pain comprises cancer-related pain.

### Embodiment B46

The method of Embodiment B33, wherein the pain is associated with lumbar nerve root compression.

### Embodiment B47

The method of Embodiment B33, wherein the pain is associated with spinal cord injury.

### Embodiment B48

The method of Embodiment B33, wherein the pain is associated with post-stroke pain.

### Embodiment B49

The method of Embodiment B33, wherein the pain is associated with central multiple sclerosis pain.

### Embodiment B50

The method of Embodiment B33, wherein the pain is associated with HIV-associated neuropathy.

### Embodiment B51

The method of Embodiment B33, wherein the pain is associated with radio-therapy associated neuropathy.

### Embodiment B52

The method of Embodiment B33, wherein the pain is associated with chemo-therapy associated neuropathy.

### Embodiment B53

The method of Embodiment B33, wherein the pain comprises dental pain.

### Embodiment B54

The method of Embodiment B33, wherein the pain is associated with primary dysmenorrhea.

### Embodiment B55

The oral dosage form of any of embodiments B24-32, comprising an amount of celecoxib that results in a celecoxib plasma level of about 1 μM to about 10 μM when the oral dosage form is administered to a human being.

### Embodiment B56

The method of any of embodiments B1-23 and B33-54, wherein celecoxib is administered at a dose that results in a celecoxib plasma level of about 1 μM to about 10 μM.

### Example 1

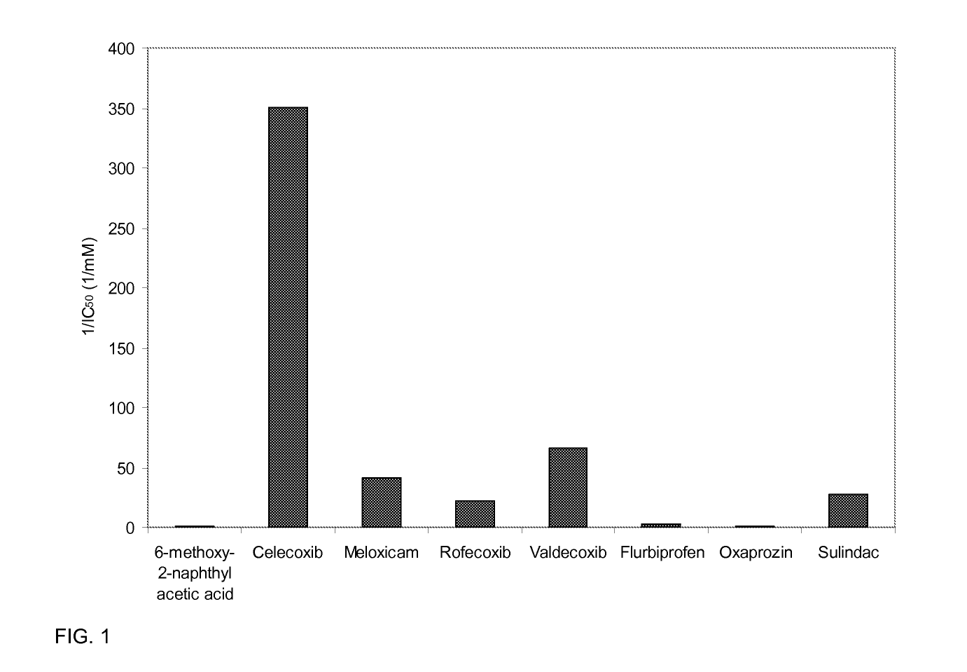

Inhibition of dextromethorphan metabolism was examined using human liver microsomes. Celecoxib was incubated at 7 different concentrations in the presence of dextromethorphan (2.5 μM) and human liver microsomes (0.5 mg/mL) at 37° C. In addition, the assay contained NADPH (2 mM), MgCl₂ (3 mM), and potassium phosphate buffer (50 mM) at pH 7.4. The assay was initiated by adding the NADPH, and incubation was terminated after 10 minutes by addition of acetonitrile. The samples were centrifuged and the metabolism of dextromethorphan was analyzed by LC/MS/MS. Results were compared to a control containing vehicle. The same assay was carried out on a number of NSAIDs. The results are summarized in Table 1 below, and depicted in FIG. 1.

TABLE 1

| Test compound | IC₅₀ (μM) |
|---|---|
| 6-methoxy-2-naphthyl acetic acid | >1760 |
| Celecoxib | 2.6, 2.9 (2 tests run) |
| Meloxicam | >24 |
| Rofecoxib | 44.5 |
| Valdecoxib | 15.2 |
| Flurbiprofen | >320 |
| Oxaprozin | >1400 |
| Sulindac | >36 |

Prior to these experiments, there was no reason to expect that celecoxib would be more active than any other NSAID in Table 1. As can be readily seen in both Table 1 and FIG. 1, celecoxib is far more active at inhibiting the metabolism of dextromethorphan than any other NSAID tested. Celecoxib is about five times as active at inhibiting the metabolism of dextromethorphan as the next best compound, and is more than 500 times as active as 6-methoxy-2-naphthyl acetic acid. This result was unexpected.

Unless otherwise indicated, all numbers expressing quantities of ingredients, properties such as molecular weight, reaction conditions, and so forth used in the specification and claims are to be understood in all instances as indicating both the exact values as shown and as being modified by the term “about.” Accordingly, unless indicated to the contrary, the numerical parameters set forth in the specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

The terms “a,” “an,” “the” and similar referents used in the context of describing the invention (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of any claim. No language in the specification should be construed as indicating any non-claimed element essential to the practice of the invention.

Groupings of alternative elements or embodiments disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, the claims include all modifications and equivalents of the subject matter recited in the claims as permitted by applicable law. Moreover, any combination of the above-described elements in all possible variations thereof is contemplated unless otherwise indicated herein or otherwise clearly contradicted by context.

In closing, it is to be understood that the embodiments disclosed herein are illustrative of the principles of the claims. Other modifications that may be employed are within the scope of the claims. Thus, by way of example, but not of limitation, alternative embodiments may be utilized in accordance with the teachings herein. Accordingly, the claims are not limited to embodiments precisely as shown and described.

1. A method of increasing dextromethorphan plasma levels in a human being that is an extensive metabolizer of dextromethorphan, comprising co-administering celecoxib with dextromethorphan to the human being.

2. A method of inhibiting metabolism of dextromethorphan, comprising administering celecoxib to a human being, wherein the human being is an extensive metabolizer of dextromethorphan, and wherein dextromethorphan is present in the body of the human being at the same time as celecoxib.

3. The method of claim 2, wherein inhibiting the metabolism of dextromethorphan causes the metabolic lifetime of dextromethorphan to be increased in the human being.

4. A method of correcting extensive metabolism of dextromethorphan, comprising administering celecoxib to a human being in need thereof.

5. The method claim 4, wherein dextromethorphan is administered to the human being for the treatment of pain.

6. (canceled)

7. (canceled)

8. (canceled)

9. (canceled)

10. (canceled)

11. (canceled)

12. (canceled)

13. (canceled)

14. (canceled)

15. The method of claim 5, wherein substantially no paracetamol is administered to the human being.

16. The method of claim 5, wherein substantially no dexamethasone is administered to the human being.

17. The method of claim 5, wherein substantially no gabapentin is administered to the human being.

18. The method of claim 5, wherein substantially no triazolam is administered to the human being.

19. The method of claim 5, wherein substantially no ondansetron is administered to the human being.

20. The method of claim 5, wherein substantially no chlorpheniramine maleate is administered to the human being.

21. The method of claim 5, wherein substantially no pseudoephedrine hydrochloride is administered to the human being.

22. An oral dosage form comprising at least 20 mg of dextromethorphan and an effective amount of celecoxib to inhibit the metabolism of dextromethorphan in a human being that is an extensive metabolizer of dextromethorphan.

23. The oral dosage form of claim 22, wherein about 30 mg to about 350 mg of dextromethorphan is present in the dosage form.

24. The oral dosage form of claim 22, wherein about 100 mg to about 400 mg of celecoxib is present in the dosage form.

25. The oral dosage form of claim 22, which is substantially free of paracetamol.

26. The oral dosage form of claim 22, which is substantially free of dexamethasone.

27. The oral dosage form of claim 22, which is substantially free of gabapentin.

28. The oral dosage form of claim 22, which is substantially free of triazolam.

29. The oral dosage form of claim 22, which is substantially free of ondansetron.

30. The oral dosage form of claim 22, which is substantially free of pseudoephedrine hydrochloride.

31. The oral dosage form of claim 22, comprising an amount of celecoxib that results in a celecoxib plasma level of about 1 μM to about 10 μM when the oral dosage form is administered to a human being.

32. The method of claim 1, wherein celecoxib is administered at a dose that results in a celecoxib plasma level of about 1 μM to about 10 μM.
